SEQUENCE LISTING (16 sequences)

SEQ ID NO: 1 - RNA, 266 nt, Homo sapiens
cucuaccugcauucccaaguaacggaaaggaguagagguuugaaucuuaucagauguuauugucagucccgcguuggcaaccucugucugcagaaauguguaacggucaauuaauuugucucucguuucugcagcacacaaaauaucaacauagugaacgauguaacagucugaaaacuugucucuaacugguugcagagcuugagcugacaaauaguugaacuauuaagcauucccugaggcaauacuuuccacugaaaccuggu

SEQ ID NO: 2 - DNA, 285 bp, Homo sapiens
accaggtttcagtggaaagtattgcctcagggaatgcttaatagttcaactatttgtcagctcaagctctgcaaccagttagagacaagttttcagactgttacatcgttcactatgttgatattttgtgtgctgcagaaacgagagacaaattaattgaccgttacacatttctgcagacagaggttgccaacgcgggactgacaataacatctgataagattcaaacctctactcctttccgttacttgggaatgcaggtagaggaaaggaaaattaaaccac

SEQ ID NO: 3 - DNA, 20 bp, Artificial Sequence
  Primer
tgcctcagggaatgcttaat

SEQ ID NO: 4 - DNA, 20 bp, Artificial Sequence
  Primer
cctctacctgcattcccaag

SEQ ID NO: 5 - RNA, 343 nt, Homo sapiens
uuaccagguugaaaugggaaacgagggagaaaggacuugaagaugacuccaguguuucuaguaacacagguggugaugucacuaaugaggguaaaagcacugaaagcgcagguaugacuuuggaaaaugguggguuugagguguuucuucccaagcaaguauuggggauucaugccaagaacuuaagagugguaccagggccagacauauaaauuugggguauuuauaucaaaugcugguagaaguagcgagauuaaaagaguuagcccugagaaaacauagagcaaggagaggcaguuaaaaucagcagagccugcugaaaaacaccuucuguagaagguag

SEQ ID NO: 6 - DNA, 343 bp, Homo sapiens
ctaccttctacagaaggtgtttttcagcaggctctgctgattttaactgcctctccttgctctatgttttctcagggctaactcttttaatctcgctacttctaccagcatttgatataaataccccaaatttatatgtctggccctggtaccactcttaagttcttggcatgaatccccaatacttgcttgggaagaaacacctcaaacccaccattttccaaagtcatacctgcgctttcagtgcttttaccctcattagtgacatcaccacctgtgttactagaaacactggagtcatcttcaagtcctttctccctcgtttcccatttcaacctggtaa

SEQ ID NO: 7 - DNA, 20 bp, Artificial
  Primer
ggtgtttttcagcaggctct

SEQ ID NO: 8 - DNA, 20 bp, Artificial
  Primer
aaaatggtgggtttgaggtg

SEQ ID NO: 9 - DNA, 21 bp, Artificial
  Primer
gattttcaccaatgaccgccg

SEQ ID NO: 10 - DNA, 17 bp, Artificial
  Primer
ccccagcattgatgtcg

SEQ ID NO: 11 - DNA, 24 bp, Artificial
  Primer
caggggaaacagacgatgacaact

SEQ ID NO: 12 - DNA, 21 bp, Artificial
  Primer
tgcggtaacccaagccacact

SEQ ID NO: 13 - DNA, 24 bp, Artificial
  Primer
gagccaaaagttcttctacactgc

SEQ ID NO: 14 - DNA, 22 bp, Artificial
  Primer
agattccagatggttctgccta

SEQ ID NO: 15 - DNA, 22 bp, Artificial
  Primer
gagatccctccaaaatcaagtg

SEQ ID NO: 16 - DNA, 22 bp, Artificial
  Primer
ccttccacgataccaaagttgt

(19) United States
(12) Patent Application Publication
(10) Pub. No.: US 2011/0045464 A1
(43) Pub. Date: Feb. 24, 2011

(54) METHODS AND COMPOSITIONS FOR IDENTIFICATION OF PROSTATE CANCER MARKERS

(75) Inventors: G. Prem-Veer Reddy, West Bloomfield, MI (US); Mani Menon, Bloomfield, MI (US)

Correspondence Address: RADER, FISHMAN & GRAUER PLLC, 39533 WOODWARD AVENUE, SUITE 140, BLOOMFIELD HILLS, MI, 48304-0610, US

(73) Assignee: Henry Ford Health System

(21) Appl. No.: 12/522,817

(22) PCT Filed: Jan. 10, 2008
(86) PCT No.: PCT/US08/50775

§ 371 Date: Jul. 19, 2010

Related U.S. Application Data
(60) Provisional application No. 60/879,634, filed on Jan. 10, 2007.

(51) Int. Cl. C12Q 1/68

(52) U.S. Cl. 435/6

(57) ABSTRACT

In some embodiments, the invention comprises methods and compositions for the assessment of prostate cancer in humans by determining the level of certain markers indicative of prostate cancer in vivo, including but not limited to SEQ ID NO:1 and SEQ ID NO:5, in tissue, blood, urine, or other biological samples.

### RELATED APPLICATIONS

This application claims priority to U.S. Provisional Patent Application No. 60/879,634 filed Jan. 10, 2007, which is hereby incorporated in full.

### STATEMENT REGARDING FEDERALLY SPONSORED RESEARCH OR DEVELOPMENT

Work underlying the invention was supported in part by NIH Grant No. RO1-DK57864 and DOD Grant No. W81XWH-05-1-0071. The government may have certain rights in the invention.

### FIELD OF THE INVENTION

The invention relates generally to the field of prostate cancer.

### BACKGROUND

Prostate cancer is the most common form of non-skin malignancy and a leading cause of cancer-related death in men in the United States. Prostate cancer generally targets men over age 50, usually with few or no symptoms of its early stages. Treatment options for prostate cancer, especially for hormone refractory prostate cancer, can be very limited.

Early detection can be important for effective treatment and management of prostate cancer. For two decades serum prostate specific antigen (“PSA”) has been used as a marker for prostate cancer detection. The advent of PSA as a biomarker has enabled early detection of prostate cancer and hence improved clinical outcome, and prostate cancer can often be found early by testing the amount of PSA in the blood. However, a low PSA level is not a guarantee of disease-free status, and an elevated PSA level is frequently associated with a negative biopsy. Moreover, elevated serum PSA lacks the specificity required to distinguish prostate cancer from other prostatic disorders, such as benign prostatic hyperplasia (“BPH”) and prostatitis (1, 2). Furthermore, PSA lacks the sensitivity to detect a large fraction of early stage tumors, since more than 15% of men with a normal serum PSA level have biopsy-proven prostate cancer (3). In addition, histological confirmation of prostate cancer requires multiple biopsies of the prostate using procedures that are too invasive to repeat at regular intervals. Finally, autopsy data from American men indicates that there is about a 49% lifetime risk of developing prostate cancer. However, the risk of having clinically detected prostate cancer in the same population is less than 18% (28), suggesting that the development and progression of prostate cancer is different in different men. Prostate cancer is a heterogeneous disease (29) whose development and progression involve changes in expression of a number of genes that determine oncogenic transformation, survival, and invasiveness of prostate cancer cells. In this context, reliable detection and prediction of outcome of the disease may benefit from identification of changes in expression of genes that influence disease development and progression.

Thus, an unmet need remains for non-invasive methods to detect markers of prostate cancer with specificity and sensitivity in biological samples, including without limitation, tissues and bodily fluids such as urine or blood.

### SUMMARY

In some embodiments, without limitation, the invention comprises methods and compositions for the identification and detection of certain molecular markers for prostate cancer with specificity and sensitivity in biological samples, including but not limited to, human prostate tissue, blood, or urine. In accordance with the invention, novel methods and compositions are provided to detect and manage prostate cancer and related indicators.

In accordance with the invention, the inventors have discovered a methodology for identifying certain particular genes expressed in human that are of particular clinical or scientific interest, as one example only, in identifying and monitoring the treatment of prostate cancer. By detection of markers for these genes at differentially elevated or lowered levels in biological samples, including but not limited to, prostate tissue, blood (including any fraction or fractions thereof), serum, or urine, detection of the presence of prostate cancer in vivo is facilitated.

In some embodiments, without limitation, unique methods and compositions allow detection of the presence of specific markers for prostate cancer in order to assess onset of prostate cancer in human subjects, as well as to monitor the response to therapy. In accordance with the invention, the presence of prostate cancer is detected by screening for expression of certain markers for one or more genes that occur at differentially elevated or suppressed levels when prostate cancer is present in the subject.

In accordance with some embodiments, the inventors adapted and applied a reverse-transcriptase polymerase chain reaction (“RT-PCR”) differential display method to first identify mRNA transcripts that are differentially expressed in tumor vs. patient-matched non-tumor prostate tissue. In doing so, the inventors discovered certain mRNA transcripts that were expressed differentially in some but not all tumor specimens examined. To identify mRNA transcripts that are differentially expressed in most tumor specimens, the inventors adapted and applied a method of differential display of pooled tissue samples, for purposes herein, described as “Averaged Differential Expression” (“ADE”). This technique was employed to assess differential display of mRNA from patient-matched non-tumor vs. tumor samples. In doing so, the inventors discovered that at least one certain mRNA transcript was over-expressed in pooled tumor RNA, as well as in the majority of individual tumor RNAs that comprised the pool. The mRNA transcript showed 100% identity to a 285 nucleotide sequence (Accession Number EH613345) in KB208E9 (Accession Number AP000345) (herein SEQ ID. NO: 1.) Similarly, based on ADE analysis, it was also discovered that at least one certain mRNA transcript was down-regulated in pooled samples as well as in the majority of individual tumor RNAs tested. The sequence of this second mRNA transcript showed 100% identity to a 343 nucleotide sequence (Accession Number EH613353) in rp11-442e11 (Accession Number AC007707.14)(herein SEQ ID NO: 5). Differential expression of these mRNA transcripts was also detected by RT-PCR in mRNA isolated from urine and blood samples of prostate cancer patients. It was also discovered that specific cDNA probes of frequently differentially expressed mRNA transcripts identified by ADE, e.g., SEQ ID NOS. 2 and 6, can be used for the detection of prostate cancer in urine and blood samples.

In some embodiments, the invention comprises the analysis of gene expression of markers for prostate cancer in order to diagnose such disorders rapidly using non-invasive urine-based tests. In one embodiment, detection of gene expression uses RT-PCR to uniquely detect SEQ ID. NO: 1 and/or SEQ ID NO: 5, or their respective corresponding nucleic acid or protein analogs, as indicators of the presence of prostate cancer in vivo. In accordance with the instant invention, these indicators become positive earlier in the course of disease than markers such as PSA and are more specific.

Without limiting the invention to only those embodiments disclosed, and without disclaiming any embodiment, in some embodiments, the invention comprises methods for assessing the presence of prostate cancer in a human, comprising the steps of (a) providing a sample of prostate tissue, blood, or urine from a human; and (b) determining the level of SEQ ID NO: 1 in the sample, wherein an elevated level of SEQ ID NO: 1 in the sample is indicative of the presence of prostate cancer in the human. Other embodiments may comprise methods for assessing the presence of prostate cancer in a human, comprising the steps of: (1) providing a sample of prostate tissue, blood, or urine from a human; and (b) determining the level of SEQ ID NO: 5 in the sample, wherein a reduced level of SEQ ID NO: 5 in the sample is indicative of the presence of prostate cancer in the sample. Still other embodiments may methods for assessing the presence of prostate cancer in a human, comprising the steps of: (a) providing a sample of prostate tissue, blood, or urine from a human; (b) determining the level of SEQ ID NO: 1 in the sample; (c) determining the level of SEQ ID NO: 5 in the sample; and (d) determining the ratio of the level of SEQ ID NO: 1 in the sample to the level of SEQ ID NO: 5 in the sample, wherein an increase in the ratio is indicative of the presence of prostate cancer in the human.

In further embodiments, the invention comprises novel primers, and kits containing same, for the detection of molecular markers of interest.

### BRIEF DESCRIPTION OF THE DRAWINGS

Some embodiments of the invention will now be described, by way of example only, with reference to the accompanying drawings, in which:

FIG. 1 shows results of mRNA RT-PCR differential display (herein “DD”) analysis of RNA from tumor vs. patient-matched non-tumor prostate tissue:

FIG. 2 shows results of averaged differential expression (“ADE”) of RNA pooled from multiple patients.

FIG. 3 shows results of RT-PCR analysis of genes identified by ADE in prostate tissue.

FIG. 4 shows results of RT-PCR analysis of KB208E9 and rp1′-442e11 mRNA in urine of prostate cancer patients.

FIG. 5 shows results of RT-PCR analysis of KB208E9 and rp11-442e11 mRNA in blood of prostate cancer patients.

FIG. 6 shows results of RT-PCR analysis of genes identified by DD.

### DETAILED DESCRIPTION

In some embodiments, without limitation, the invention comprises the identification and analysis of one or more markers for gene sequences that are indicative of the presence of prostate cancer in vivo in human subjects.

In accordance with some embodiments, the inventors adapted and applied an RT-PCR differential display method to first identify mRNA transcripts differentially expressed in tumor vs. patient-matched non-tumor prostate tissue. By doing so, 44 mRNA transcripts were identified that were expressed differentially in some but not all of the tumor specimens examined.

To identify mRNA transcripts that were differentially expressed in most tumor specimens, the inventors adapted and applied a method of differential display of pooled tissue samples, designated “Averaged Differential Expression” (“ADE”). Differential display of mRNA was performed from patient-matched non-tumor vs. tumor tissue, each pooled from ten patients with various Gleason scores. The results showed that differentially expressed mRNA transcripts identified by ADE were fewer in number than by DD, but were expressed in a greater percentage of tumors (>75%) than those identified by differential display of mRNA from individual patient samples. Differential expression of these mRNA transcripts was also detected by RT-PCR in mRNA isolated from urine and blood samples of prostate cancer patients.

Such findings support the inventors' concept that specific cDNA probes of frequently differentially expressed mRNA transcripts identified by ADE can be used for the detection of prostate cancer in biological samples, including without limitation, in urine and blood samples. Thus, Differential Display (DD) (4, 5) was used to investigate and identify mRNA transcripts that are expressed differentially in tumor compared to matched non-tumor prostate tissues from patients who underwent radical prostatectomy. DD analysis is generally known to those of ordinary skill in the relevant art (4, 5, 6, 7). This requires only small amounts of starting RNA and can support rapid identification of over-expressed and down-regulated messages and low abundance mRNAs that are involved in regulatory processes of the cell (8).

In the inventors' work. DD analysis of individual tumors provided information on a number of genes, but the differential expression of several of these genes could be verified by RT-PCR in less than 20% of tumors. The use of DD to compare pooled tumors vs. their pooled non-tumor contra-lateral prostate specimens was further investigated in order to assess whether this method would reveal genes differentially expressed in the majority of samples. This DD of pooled tumors is referred to herein as ADE. Results of testing showed that ADE identified fewer genes than DD of individual tumors; however, their expression was confirmed in >75% of the tumors under study. Furthermore, it was discovered that gene changes identified by ADE were readily detectable in urine and blood of patients with advanced prostate cancer.

Thus, in accordance with some embodiments of the invention, ADE supports the identification of genes whose expression is altered in a wide population of patients with a heterogeneous cancer such prostate cancer. Similarly, the relative levels of over-expressed and down-regulated genes identified in body fluids provide a viable option for reliable and early detection of prostate cancer.

The following examples of embodiments of the invention are provided without limiting the scope of the invention to only those embodiments disclosed herein and without disclaiming any other embodiments:

### EXAMPLES

### Materials and Methods

Tissue specimens: Prostate tumors were obtained from human radical prostatectomy specimens. None of the patients included in the study had received hormonal therapy, chemotherapy, or radiation therapy. The protocol was reviewed and approved by an appropriate Institutional Review Board. Cancerous tissues were graded by a pathologist according to the Gleason scoring system. Non-tumor prostate tissue was obtained from the contra-lateral lobe of the same specimen. Cancer and matched non-tumor tissues were stored frozen at −80° C. within an hour of surgical excision.

Blood and urine specimens: Peripheral blood and urine samples were obtained from prostate cancer patients undergoing chemotherapy. Blood was collected in PAXgene blood RNA tubes for RNA stabilization (Qiagen, Valencia, Calif.). These tubes were stored at RT for at least 2 hours before RNA isolation was performed. Urine was collected in an equal volume of Lysis Buffer containing 5.64 M guanidinium thiocyanate, 0.5% sarcosyl, 50 mM sodium acetate (pH 6.5) and 1 mM β-mercaptoethanol, and the pH was adjusted to 7.0 with 1.5 M HEPES (pH 8.0); these samples were frozen at −80° C. until extraction of RNA was performed. This procedure allows recovery of total RNA (both intra- and extra-cellular) in urine. All patients provided written informed consent, and protocols were approved by an appropriate Institutional Review Board.

RNA isolation: Total RNA was extracted from frozen prostate tissue specimens with RNeasy Mini Kit (Qiagen, Valencia, Calif.) according to the manufacturer's protocols. For isolation of total cellular RNA from blood, PAXgene Blood RNA Kit was used (Qiagen, Valencia. CA). Isolation of RNA from urine was carried out using, the protocol of Menke and Warnecke (39). DNA was removed by performing on-column DNase digestion with RNase-free DNase (Qiagen, Valencia, Calif.). The integrity and size distribution of RNA was monitored by agarose gel electrophoresis.

RT-PCR differential display (DD): DD was performed by using the RNAimage Kit (GenHunter. Nashville, Tenn.) as described by Liang and Pardee (5). RNAs isolated from tumor and matched non-tumor prostate tissues obtained from the same surgical specimen were compared by DD. RT-PCR for DD of individual surgical specimens was performed using 24 different primer pair combinations involving 3 anchor primers (H-T11C, H-T11G, and H-T11A) and 8 arbitrary primers (H-AP17 to H-AP24) from GenHunter (Nashville. TN). RT-PCR for DD of pooled surgical specimens from multiple patients (ADE) was performed using anchor primer H-T11C and arbitrary primer H-AP17. Reverse transcription of 200 ng of individual or pooled RNA was performed with Sensiscript RT (Qiagen, Santa Clarita, Calif.). Reactions containing 2 μl 10×RT buffer, 2 μl 5 mM dNTP (final concentration 500 μM), 2 μl 10 μM anchor primer (final concentration 1 μM), 2 μl RT, 1 μl RNase Inhibitor (10 U/μl) and 10 μl RNase-free water were incubated at 37° C. for 30 min and then at 93° C. for 5 min. 10% of the RT reaction was used for subsequent PCR, in duplicate. The PCR reaction contained 200 nM each of anchor primer and arbitrary primer (e.g., H-T11C and H-AP19, or H-T11C and H-AP17), 10 mM Tris-Cl, pH 8.4, 50 mM KCl, 1.5 mM MgCl₂, 5 mM DTT. 2 μM dNTP mix, 20 Ci/mmol [α-³³P]dATP and 2 U Tag Polymerase (Qiagen, Santa Clarita, Calif.) in a total volume of 20 μl. The cycling parameters were 94° C. for 15 sec. 40° C. for 2 min and 72° C. for 30 sec followed by 72° C. for 5 min. Forty PCR cycles were performed for amplification of RNA from both tumor and patient-matched non-tumor tissues. PCR products were subjected to denaturing 6% polyacrylamide gel electrophoresis on an extended format using programmable Genomyx LR gel electrophoresis apparatus (Beckman Coulter, Columbia, Md.). cDNA bands that were either more abundant or less abundant in tumor than in non-tumor RNA were excised, re-amplified using the same primers used for DD, and sequenced directly or after cloning into pGEM-T vector (Invitrogen, Carlsbad, Calif.), as described (5). Clones were screened for the insert and then sequenced. Sequences of differentially expressed mRNA transcripts were then searched for homology to known gene sequences in GenBank using the BLAST algorithm (40).

RT-PCR analysis of differentially expressed genes: In order to confirm differential expression of genes identified by DD, semi-quantitative RT-PCR was performed using primers based on the sequence of the DD cDNA fragments. These primer sequences were 5′-GATTTTCACCAATGACCGCCG (forward) (SEQ ID NO: 9) and 5′-CCCCAGCATTGATGTCG (reverse) (SEQ ID NO: 10) for TRPM8, 5′-CAGGGGAAACAGACGATGACAACT (forward) (SEQ ID NO:11) and 5′-TGCGGTAACCCAAGCCACACT (reverse) (SEQ ID NO:12) for ADAMTS9, 5′-GAGCCAAAAGTTCTTCTACACTGC (forward) (SEQ ID NO: 13) and 5′-AGATTCCAGATGGTTCTGCCTA (reverse) (SEQ ID NO: 14) for RP11-571N1, 5′-TGCCTCAGGGAATGCTTAAT (forward) (SEQ ID NO: 3) and 5′-CCTCTACCTGCATTCCCAAG (reverse) (SEQ ID NO: 4) for KB208E9, 5′-GGTGTTTTTCAGCAGGCTCT (forward) (SEQ ID NO: 7) and 5′-AAAATGGTGGGTTTGAGGTG (reverse) (SEQ ID NO: 8) for rp11-442e11, and 5′-GAGATCCCTCCAAAATCAAGTG (forward) (SEQ ID NO: 15) and 5′-CCTTCCACGATACCAAAGTTGT (reverse) (SEQ ID NO: 16) for GAPDH. cMaster RTₚₗᵤₛ PCR system (Brinkman Instruments Inc, Westbury, N.Y.) was used to reverse transcribe and amplify total RNA from tissue, blood or urine. RNA was reverse-transcribed using oligo (dT) primer and cMaster reverse-transcriptase according to the manufacturer's protocol. The enzyme was inactivated for 5 minutes at 85° C. and cDNA was stored at −80° C. until use. Amplification of cDNA was carried out using primers described above for each gene. Different PCR cycle numbers were tested for each gene to ensure that the assay was in the linear range of amplification. The constitutively expressed housekeeping gene GAPDH was amplified from each sample to normalize the level of each test gene. PCR products were run on a 2% agarose gel. Quantitation was carried out by digital analysis of band intensity in the gel with an Eagle Eye II Still Video System, using the EagleSight software (version 3.2; Stratagene, La Jolla, Calif.).

Results

Identification of genes differentially expressed between tumor and non-tumor prostate tissue from radical prostatectomy patients: To attempt to identify biomarkers for prostate cancer detection, DD was performed on tumor and matched non-tumor prostate tissues from prostatectomy patients to investigate differences in expression of numerous genes (5). DD was performed on tissues from 7 patients representing Gleason grades 3+3 (3 patients). 3+4 (1 patient). 4+4 (2 patients) and 5+4 (1 patient), using 24 different anchor and arbitrary primer sets for cDNA amplification. Using this method, the inventors identified 286 differentially expressed cDNA bands (191 over-expressed and 95 down-regulated). Of these 286 bands, 44 (37 over-expressed and 7 down-regulated) have been extracted from the gels and sequenced to date. The Accession Number and gene identity of each of these sequences is presented in Table 1 herein.

TABLE 1
Differentially expressed genes in prostate cancer identified
by DD RT-PCR analysis of individual tumors.

| | | |
|---|---|---|
| No | Accession Number | Name |

mRNA transcripts over-expressed in prostate cancer

| | | |
|---|---|---|
| 1 | NM_024080.3 | TRPM8 |
| 2 | NG_001336.2 | T cell receptor gamma locus |
| 3 | BC050454.1 | Dishevelled, dsh homolog 1 |
| 4 | BC016066.1 | Calpastatin mRNA |
| 5 | BC032297.1 | Tripartite motif containing 26, mRNA |
| 6 | NM_001206.1 | BTEB1 |
| 7 | HS353E16 | PITPNB for phosphatidylinositol transfer |
| | | protein beta |
| 8 | AF483622 | RFC2 |
| 9 | AK023672.1 | Cisplatin resistance associated over-expressed |
| | | protein mRNA |
| 10 | AC104805.3 | RP11-571N1 |
| 11 | AC026224.6 | RP11-74N14 |
| 12 | AC106878.3 | RP11-54K16 |
| 13 | AC106878.3 | RP11-64809 |
| 14 | AC093619.5 | RP13-741A20 |
| 15 | AC104164.2 | RP11-641C17 |
| 16 | AC055733.16 | RP11-39E3 |
| 17 | AC116098.3 | CTD-2329B17 |
| 18 | AL117329.8 | RP11-191L9 |
| 19 | BC008696.1 | Clone image 2820627 |
| 20 | AC109486.2 | RP11-546M4 |
| 21 | AC094086.2 | CTD-2170G1 |
| 22 | AL139194.7 | BAC C-2190G12 |
| 23 | AC058791.4 | RP11-138A9 |
| 24 | BC041856.1 | Clone image 5270501 |
| 25 | HS171N11 | RP1-171N11 |
| 26 | AC007032.2 | RP11-22N19 |
| 27 | AC093752.2 | RP11-33B1 |
| 28 | AC117984.2 | CTD-2503H21 |
| 29 | AC012598.16 | RP11-237K10 |
| 30 | AY166681.1 | RP4-76112 on Chr 6 |
| 31 | AC069506.14 | RP11-321G3 |
| 32 | AL513328.12 | RP13-461N9 |
| 33 | AK092048.1 | cDNA FLJ34729 fis, Clone MESAN20064 |
| 34 | AC090527.3 | RP11-96020 |
| 35 | AC080094.5 | RP11-1007E2 |
| 36 | AL122001.32 | RP4-603114 |
| 37 | AC087525.6 | RP11-321G12 |

mRNA transcripts down-regulated in prostate cancer.

| | | |
|---|---|---|
| 1 | NM_020249 | ADAMTS9 |
| 2 | BC009175.2 | EBNA1 binding protein mRNA |
| 3 | AL096710 | BPAG1 |
| 4 | AF263545 | HUT11 protein mRNA |
| 5 | HSJ300O13 | RP1-300013 |
| 6 | AC108709 | 3BAC RP11-81P15 |
| 7 | AC011295 | BAC RP11-96J23 |

Of these 44 sequenced mRNAs, only 13 matched mRNA sequences in GenBank; the rest were expressed sequence tags (“ESTs”) that had not been reported previously. Thus, in accordance with the inventors' work, by applying, DD to tumor and patient-matched non-tumor prostate tissue, a number of new mRNA transcripts were discovered.

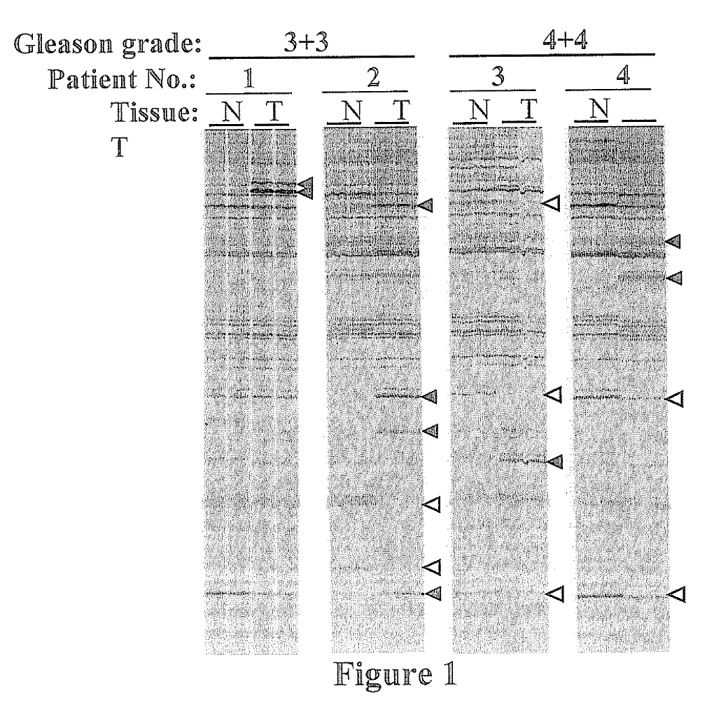

FIG. 1 shows results of a representative DD of RNA amplified from tumor vs. patient-matched non-tumor prostate tissue from 4 different patients using the same anchor and arbitrary primer set (H-T11C and H-AP17). RNA was isolated from prostate tumor (“T” for “tumor”) and matched non-tumor (“N” for “non-tumor”) prostate tissue from individual patients, and reverse-transcribed with anchor primer H-T11C. The resultant cDNA was amplified with primer H-T11C and arbitrary primer H-AP19 as described in the Materials and Methods. The PCR reactions for each sample were run in duplicate. The amplified products were separated on an extended format 6% polyacrylamide gel. Differentially expressed mRNA transcripts in individual patients are indicated by arrowheads; closed arrowheads indicate over-expressed mRNA transcripts, and open arrowheads indicate down-regulated mRNA transcripts in tumor, as compared to non-tumor, prostate tissue from individual patients. Tumors of patients 1 and 2 were of Gleason grade 3+3, and those in patients 3 and 4 were of Gleason grade 4+4.

DD performed on different days with the same tissue samples using the same anchor and arbitrary primer pairs yielded essentially the same profile (data not shown). Most of the bands were of similar intensity in matched tumor and non-tumor RNA. However, bands differentially expressed in one tumor/non-tumor pair were not necessarily differentially expressed in other tumor/non-tumor pairs. For example, even tumors with the same Gleason grade differed (compare differentially expressed cDNA bands identified by arrowheads in patients 1 versus 2, both with Gleason grades 3+3, and patients 3 versus 4, both with Gleason grades 4+4).

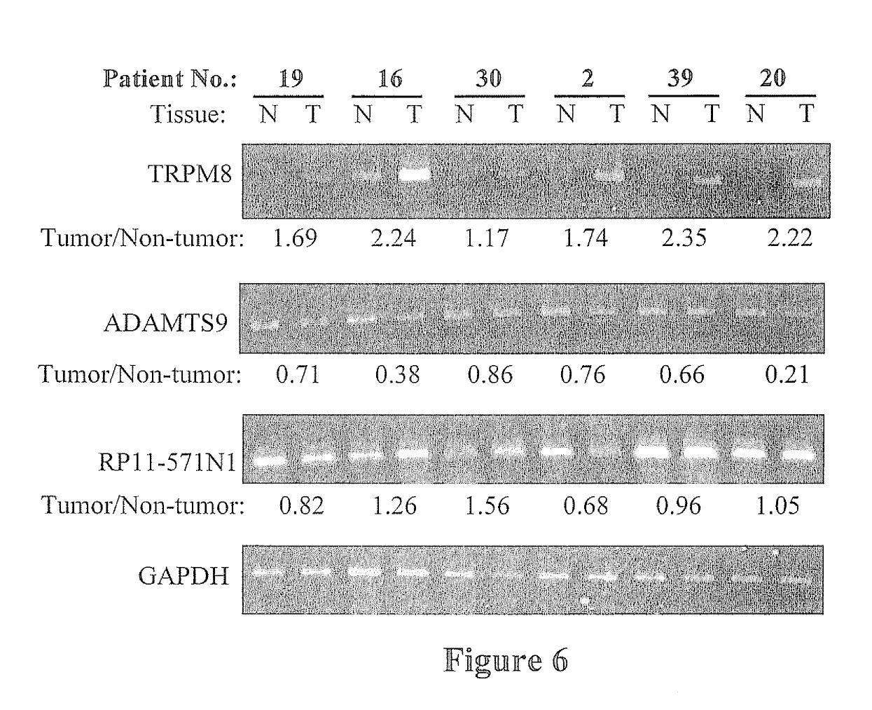

Of the 44 transcripts listed in Table 1, most were differentially expressed in only one of seven tumors and therefore were not studied further by RT-PCR to evaluate changes in a cross-section of patients. However, a few transcripts were differentially expressed in multiple tumor/non-tumor pairs, and these were analyzed further by RT-PCR with gene specific primers, using RNA isolated from another set of tumor/non-tumor pairs. FIG. 6 shows results of RT-PCR analysis of certain genes identified by DD. TRPM8 (Panel A). ADAMTS9 (Panel B) and RP11-571N1 (Panel C) transcript levels in prostate tumor (“T”) and patient-matched non-tumor (“N”) prostate tissue were analyzed by RT-PCR using gene-specific primers described in Materials and Methods. GAPDH was included as a housekeeping gene. Band intensities were quantified by densitometry, normalized to GAPDH, and expressed below each panel as a ratio of the transcript level in tumor vs. non-tumor. Patients 19, 16, 30, 2, 39, and 20 had tumors of Gleason grade 3+3, 3+4, 4+3, 4+4, 4+4, and 5+4, respectively.

TRPM8 was found by DD to be over-expressed in 3 of 7 tumors, and RT-PCR confirmed over-expression (>1.5-fold) in another 5 of 6 tumors (FIG. 6). By comparison, in the same tumors, ADAMTS9 was down-regulated (<0.5-fold) in 2 of 6 tumors, and RP11-571N1 was up-regulated (>1.5-fold) in one of six tumors, frequencies comparable to those found by DD. Thus, DD data correlated with RT-PCR data, and DD showed sensitivity to detect low abundance transcript differences in individual patient samples.

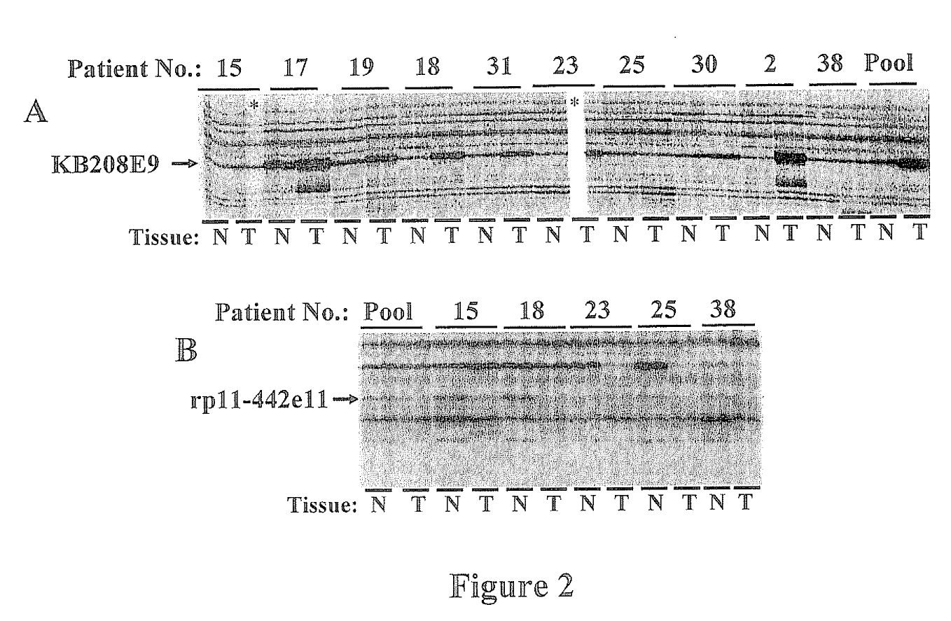

Identification of mRNA transcripts that can detect prostate cancer in a majority of patients using ADE: In order to attempt to increase the odds of identifying transcript differences common to a majority of tumor/non-tumor pairs, DD was carried out using RNA pooled from multiple patients (pooled tumor RNA versus pooled non-tumor RNA). ADE being the term for DD of RNA pooled from multiple patients. As summarized in the results of FIG. 2, RNA was isolated from tumor and patient-matched non-tumor prostate tissues. DD was performed on individual tumor-non-tumor pairs or on pooled tumor vs. pooled non-tumor, using anchor primer H-T11C and arbitrary primer H-AP17. Two DD profiles of pooled RNA revealed one band higher in tumor in 7 of 10 individual tumor/non-tumor pairs and another band lower in 3 of 5 tumor/non-tumor pairs, respectively. These bands were identified as KB208E9 and rp11-442e11, based on their excision, cloning, sequencing, and BLAST analysis in accordance with methods known to those of ordinary skill. The Gleason grade of the tumors used in our study were 3+3 (patients 15, 17, and 19), 3+4 (patients 18, and 31), 3+5 (patient 23), 4+3 (patients 25 and 30), and 4+4 (patients 2 and 38). [In FIG. 2, “N” non-tumor tissue; “T”=tumor tissue.]

ADE analysis of RNA pooled from 10 different patient specimens (tumor vs. non-tumor) led to our discovery of an mRNA transcript that was over-expressed in the pooled tumor RNA, as well as in seven of the ten individual tumor RNAs that comprised the pool (FIG. 2A). The sequence of this mRNA transcript showed 100% identity to a 285 nucleotide sequence in KB208E9 (Accession Number AP000345). Based on another ADE analysis of RNA pooled from 5 patient specimens (tumor vs. non-tumor), we also discovered the down-regulation of an mRNA transcript in pooled, as well as in three of the five individual, tumor RNAs (FIG. 2B). The sequence of this mRNA transcript showed 100% identity to a 343 nucleotide sequence in rp11-442e11 (Accession Number AC007707.14). These two were the only differentially expressed transcripts that were identified by ADE with the one primer pair used.

Sequences of mRNA transcripts identified by the inventors as described herein have been deposited in the GenBank database.

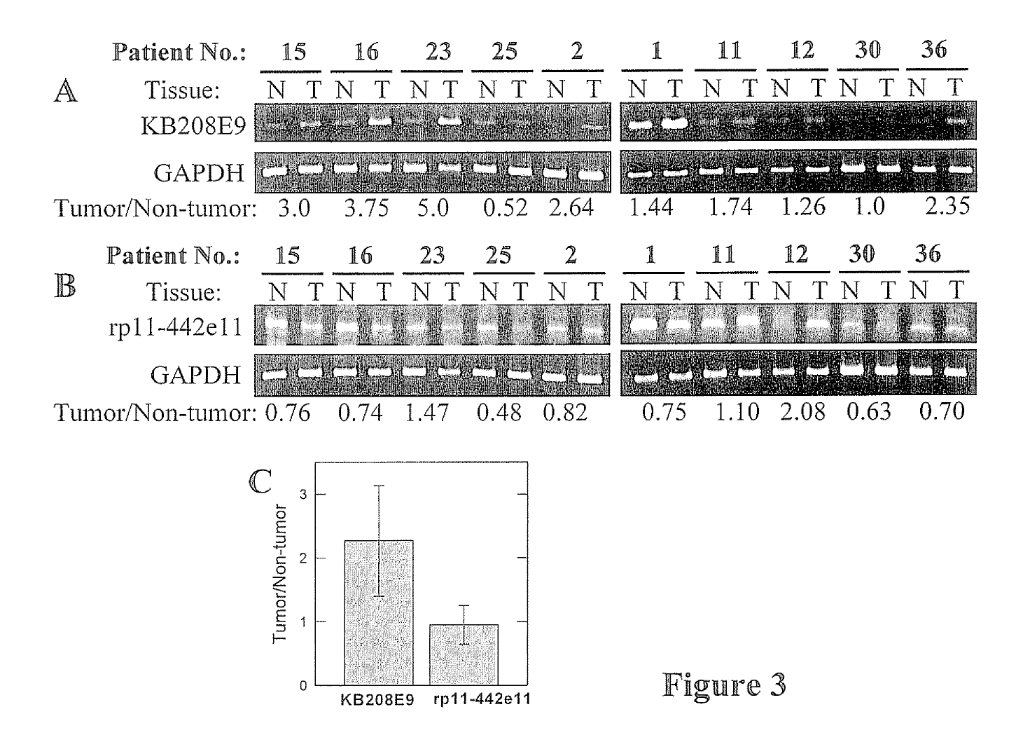

RT-PCR validation of differential expression of KB208E9 and rp11-442e11 in prostate tissue from cancer patients: In order to confirm differential expression of genes identified by ADE, RT-PCR with gene-specific primers was used to measure KB208E9 and rp11-442e11 transcript levels in tumor vs. non-tumor pairs from 19 patients. FIG. 3 shows results of RT-PCR using gene-specific primers to analyze the levels of KB208E9 (Panel A) and rp11-442e11 (Panel B) mRNA in tumors and matched non-tumor prostate tissue. GAPDH was included as a housekeeping gene. KB208E9 and GAPDH were amplified using 25 cycles; rp11-442e11, present at lower levels, was amplified using 30 cycles. The number of PCR cycles used for each of these transcripts was determined to be in a linear range for semi-quantitative analysis. KB208E9 (Panel A) and rp11-442e11 (Panel B) were quantitated by densitometry, normalized to GAPDH, and expressed as a ratio in tumor vs. non-tumor (number below each panel). Panel A and B illustrate data from 10 tumor-non-tumor pairs. Panel C summarizes data from these 10 patients plus an additional 9 patients.

Representative RT-PCR results from tissues (tumor vs. non-tumor) of 10 of the 19 patients are presented in FIGS. 3A and 3B. We discovered that KB2088E9 was over-expressed in 13 and rp11-442e11 was down-regulated in 12 of these 19 patients. The mean tumor/non-tumor ratio of the KB208E9 transcript, normalized to GAPDH, in 19 patients was 1.96±0.263, and the mean tumor/non-tumor ratio of rp11-442e11 was 0.89±0.09 (p=0.01) (FIG. 3C). Since both transcripts were analyzed in each tumor vs. non-tumor pairs, the ratio of these transcripts was calculated; the mean ratio of KB208E9/rp11-442e11 was 2.13±0.27 (n=19). These data indicate that the ratio of KB208E9 to rp11-442e11 can be of diagnostic value.

Detection of KB208E9 and rp11-442e11 in blood and urine of prostate cancer patients: We also investigated whether in RNA transcripts identified by ADE could be detected in body fluids. Blood and urine samples were obtained from nine patients (Table 2 below) undergoing treatment for disseminated prostate cancer.

TABLE 2
Characteristics of prostate cancer patients whose urine and blood
specimens were analyzed for KB208E9 and rp11-44e11 levels.

| | PSA | | Treatment | |
|---|---|---|---|---|
| Patient | ng/ml | Gleason Score | received | Disease status |
| A | 27.4 | 7 | ADT | Rising PSA |
| B | 6.6 | 9 | Chemo | Metastatic |
| C | 1.0 | 8 | Radiation, ADT | Rising PSA |
| D | <0.2 | 6 | ADT | In remission |
| E | <0.2 | 8 | ADT, Radiation | In remission |
| F | 179.4 | Not known | Radiation, Chemo | Metastatic |
| G | 398.6 | 9 | Chemo | Metastatic |
| H | 5.3 | 7 | ADT | Metastatic |
| I | 5.4 | 6 | Radiation | Biochemical relapse |

(ADT, Androgen-deprivation therapy; Chemo, chemotherapy; Radiation, radiation therapy.)

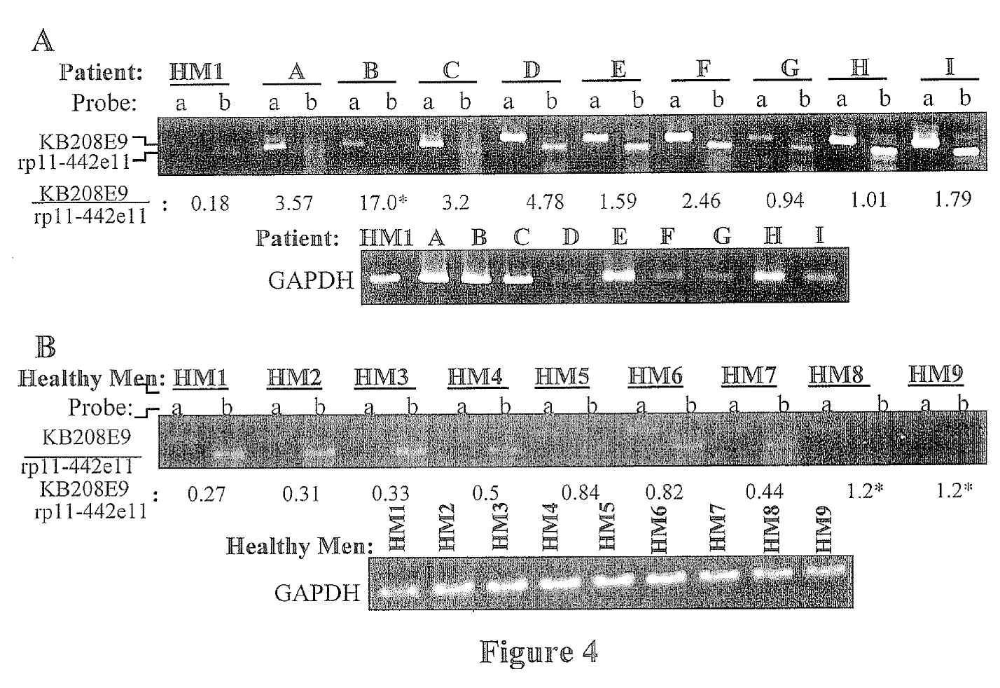

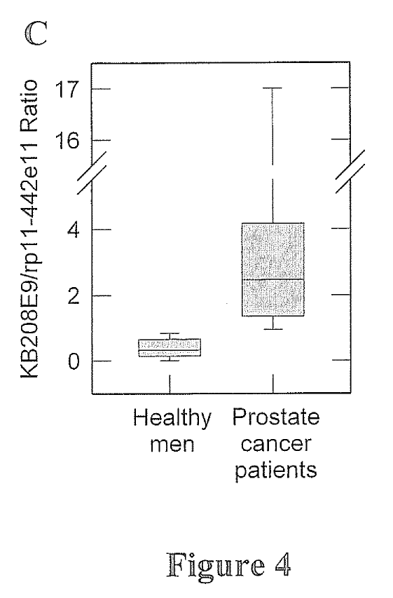

Blood and urine specimens from nine healthy men were used as controls. RNA was prepared from blood and urine and analyzed for KB208E9, rp11-442e11, and GAPDH transcript levels by RT-PCR using gene-specific primers. As represented by the results shown in FIG. 4, RNA was isolated from individual urine specimens, and RT-PCR performed with sequence specific primers for KB208E9. rp11-442e11, and GAPDH. PCR reactions were performed for 30 cycles. Numbers below each panel represent the ratio of KB208E9 to rp11-442e11, based on densitometry. GAPDH is shown as an indicator of RNA in each sample. Panel A shows the level of KB208E9 (probe a) and rp11-442e11 (probe b) in the urine RNA of a healthy man (HM1) and 9 prostate cancer patients (A to I). Panel B shows the level of KB208E9 (probe a) and rp11-442e11 (probe b) in urine RNA of nine healthy men (HM1-HM9). Panel C shows the mean ratio of KB208E9 to rp11-442e11 in healthy men (0.66±0.12, n=9) vs. prostate cancer patients (4.04±1.67, n=9). [*, Approximate value; a more reliable value could not be obtained because of low rp11-442e11 levels in the sample.]

As shown in FIG. 4, KB208E9 (lanes labeled probe a) and rp11-442e11 (lanes labeled probe b) transcript levels were substantially higher in the urine of patients (FIG. 4A) than of healthy men (FIG. 4B). Most noticeably, the ratio of KB208E9 to rp11-442e11 in urine was 4- to 5-fold higher in prostate cancer patients (4.04±1.67, n=9) than in healthy men (0.66±0.12. n=9) (FIG. 4C).

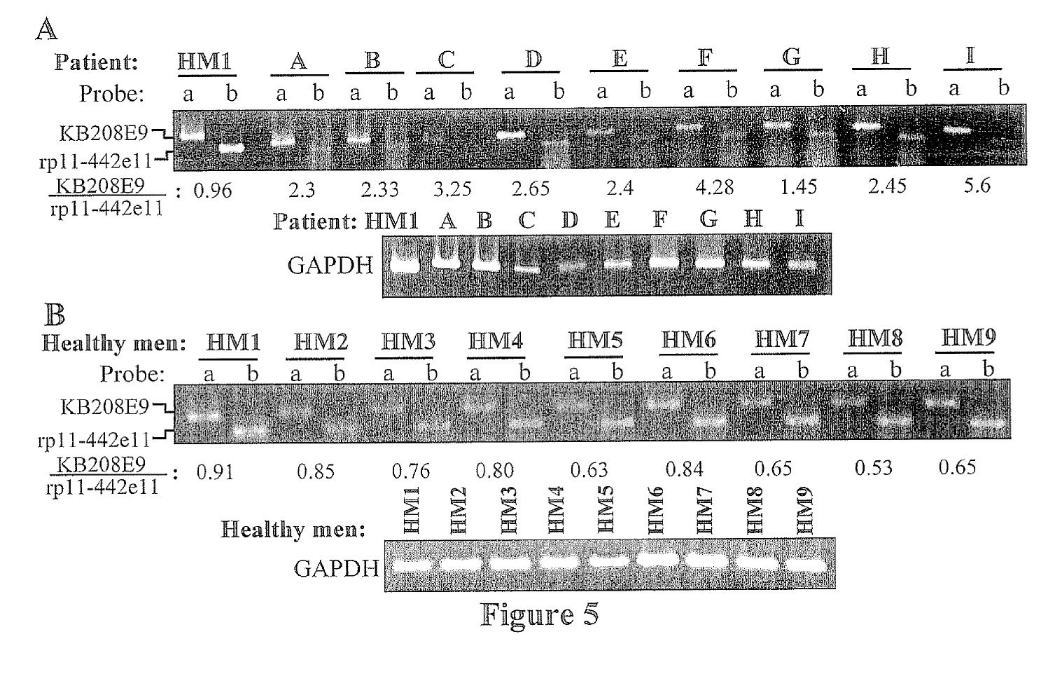

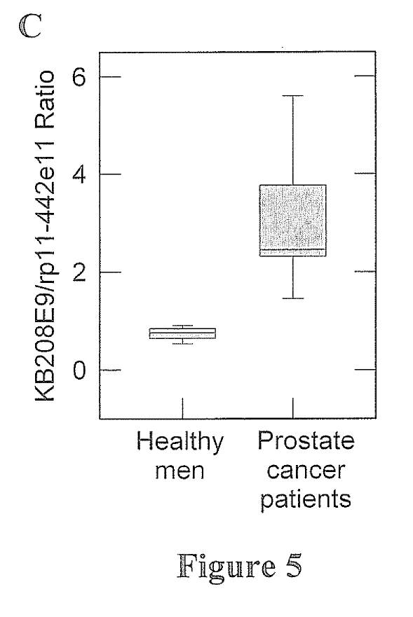

KB208E9 and rp11-442e11 transcripts were also detected in the blood of these subjects (FIG. 5). RNA was isolated from individual blood specimens and RT-PCR was performed with sequence specific primers for KB208E9, rp11-442e11 and GAPDH. PCR reactions were performed for 30 cycles. Numbers below each panel represent the ratio of KB208E9 to rp11-442e11, based on densitometry. GAPDH is shown as an indicator of RNA per sample. Panel A shows the level of KB208E9 (probe a) and rp11-442e11 (probe b) in blood RNA of one healthy man (HM1) and 9 prostate cancer patients (A to I). Panel B shows the level of KB208E9 (probe a) and rp11-442e11 (probe b) in blood RNA of nine healthy men (HM1-HM9). Panel C shows the mean ratio of KB208E9 to rp11-442e11 in healthy men (0.74±0.04, n=9) and prostate cancer patients (2.97±0.42, n=9).

The ratio of KB208E9 to rp11-442e11 was 2.97±0.42 (n=9) in the prostate cancer patients (FIG. 5A) vs. 0.74±0.42 (n=9) in the blood of the healthy men (FIGS. 5B, 5C). Thus, the ratio of KB208E9 to rp11-442e11 in both urine and blood was 4- to 5-fold higher in prostate cancer patients than in healthy men (FIGS. 4C and 5C).

No difference was found in the level of expression of PSA mRNA between tumor vs. non-tumor tissue specimens from prostate cancer patients (data not shown). It is reported that quantitative RT-PCR showed no difference in PSA mRNA levels between blood samples from patients with localized prostate cancer and healthy men (9). Also, no significant difference was observed in PSA mRNA levels between blood samples of patients undergoing treatment for disseminated prostate cancer and healthy men (data not shown). Furthermore, as shown in Table 2, there were also some prostate cancer patients (patients D and E) on androgen-deprivation therapy (ADT) and/or radiation therapy in whom serum PSA levels were below 0.2 ng/ml, yet had detectable levels of KB208E9 in their urine and blood.

Consistent with our work and discoveries, some embodiments of the present invention, without limitation, comprise unique methods and compositions that allow detection of the presence of specific markers indicative of prostate cancer in vivo in order to assess onset of prostate cancer in human subjects, as well as to monitor the response to therapy. Using adapted DD technique, we discovered mRNA transcripts that are expressed differentially in many individual tumors as compared to matched non-tumor prostate tissues from patients who underwent radical prostatectomy. Our identification of 44 differentially expressed mRNA transcripts of which 31 were novel (Table 1). Thus, the majority of the DD mRNA transcripts identified in our study are novel at least in the sense that they do not correspond to transcripts previously deposited in GenBank. The few DD mRNA transcripts that matched GenBank transcripts are reported to be altered in a variety of cancer types.

Particularly noteworthy among the mRNA transcripts that matched sequences in GenBank were TRPM8 and ADAMTS9. TRPM8 was over-expressed and ADAMTS9 was down-regulated in tumors from over 70% of the prostate cancer patients examined (FIG. 6). TRPM8 is a member of the transient receptor potential (TRP) family of Ca⁺⁺-channel proteins that is reported to be androgen-regulated and required for the survival of prostate cancer cells (10), and over-expressed in several cancers including prostate, breast, colorectal and lung (11). ADAMTS9 belongs to a subgroup of the “a distinctive and metalloproteinase with thrombospondin motifs” (ADAMTS) family of enzymes capable of cleaving versican (chondroitin sulphate proteoglycan-2). Increased expression of versican is associated with the local spread of tumor cells, potentially via destabilization of focal adhesion (12). Down-regulation of ADAMTS9 therefore can result in the accumulation of versican in the stromal compartment of the prostate (13). Our observation that ADAMTS9 is down-regulated in prostate tumor tissue is consistent with such a possibility.

The expression profile of most of the genes identified in our work varied from patient to patient (FIG. 1), in part due to the heterogeneous nature of the disease, and in part due to admixture of tumor cells with non-tumor cells. The differential expression of some of these genes could be verified by RT-PCR in less than 20% of tumors. Thus, genes identified by DD of an individual tumor provide information on the expression profile of that individual, but in our work were not themselves determinative of a profile common to all prostate cancer patients.

Our work lead to our discovery that a profile common to most prostate cancer patients can be obtained by performing DD on pooled RNAs from multiple patients' tumor and matched non-tumor prostate tissues. Differentially expressed mRNA transcripts identified by ADE were expressed in a greater percentage of tumors (>70%) than those identified by DD of mRNA from individual patient samples, and were fewer in number.

In our work, we discovered with one primer combination that two genes, KB208E9 and rp1′-442e11, were differentially expressed in more than 70% of the prostate cancer tumors tested. KB208E9 was elevated in tumor tissues of most patients who underwent radical prostatectomy irrespective of whether they presented with Gleason grade 3, 4, or 5 disease (FIG. 2). A differentially expressed cDNA sequence of 285 nucleotides showed 100% homology to a portion of genomic sequence (clone KB208E9, Accession Number AP000346.1, at Chr22q11.2) that contains no known genes or ESTs. It also had 97% identity with a 277 bp region of human endogenous retrovirus K (HERV-K) mRNA (Accession Number U39937), implicated in certain cancers (14), and a recent study has shown the presence of HERV-K mRNA in human breast cancer cell lines (15). Another cDNA sequence of 343 nucleotides showed 100% homology to a portion of genomic sequence (clone rp1′-442e11, Accession Number 007707.14, at chr 11q23.3) that corresponds to intron 4 of the RefSeq gene KIAA0999 (http://genome.ucsc.edu). Thus it appears that prostate cancer expresses decreased levels of an alternate splice variant of KIAA0999 that has not been identified previously.

Thus, in our work, whereas DD in general allowed the detection of novel and low-abundance mRNA transcripts with altered expression in individual patients, ADE identified uncommon mRNA transcripts whose expression is altered in most of the patients.

For two decades early detection of prostate cancer and hence improved clinical outcome can be attributed to the advent of prostate specific antigen (PSA) in serum as a biomarker. However, a low PSA is not a guarantee of disease-free status, and elevated serum PSA lacks the specificity required to distinguish prostate cancer from other prostatic disorders. We observed no difference in the level of expression of PSA mRNA between tumor vs. non-tumor tissue specimens from prostate cancer patients (data not shown).

Circulating epithelial cells in cancer patients permit detection of DNA- (16), protein-(17), and RNA- (18) based prostate cancer markers. It is evident from biochemical recurrence in nearly 25% of patients who have undergone radical prostatectomy for organ-confined prostate cancer (19) that tumor cells can escape from the primary site into the circulation during very early stages of the disease. Prostate epithelial cells indeed have been found in the blood of patients diagnosed with prostate cancer (2, 20-22). It is also evident that at an early stage localized primary tumors may harbor cells with metastatic potential, and exhibit a gene-expression signature matching that observed in metastatic colonies (23, 24). Some genes that are increased in prostate cancer tissue (25, 26) are also found to be elevated in patient urine (27). Thus, cancer cells that enter the circulation even during early stages of tumor growth might display characteristics of cancer that is either likely to metastasize or remain indolent. Therefore we have focused on and accomplished the discovery of certain molecular markers that are sensitive and specific enough to detect prostate cancer in easily obtainable body fluids such as blood and urine.

Thus, in accordance with some embodiments of the inventions, without limitation, certain gene expression changes identified by ADE were readily detectable by RT-PCR of mRNA isolated from urine and blood of patients undergoing treatment for disseminated prostate cancer; KB208E9 and rp11-442e1 were present at different levels in urine and blood of prostate cancer patients relative to healthy men, and the ratio of KB208E9 to rp11-442e11 was 3- to 4-fold higher in prostate cancer patients (FIGS. 4 and 5); an increase in KB208E9 levels was observed in all patients irrespective of whether the disease was in remission (patients undergoing ADT and/or radiation therapy for biochemical recurrence after radical prostatectomy) or hormone-refractory (metastatic patients undergoing chemotherapy); and the KB208E9/rp11-442e11 ratios of prostate cancer patients compared to healthy men show little or no overlap (FIGS. 4 and 5). Thus, we have discovered that increased KB208E9, reduced rp11-442e11, and/or increased ratio of KB208E9/rp11-442e11 can characterize patients with localized and advanced disease. Our discoveries support the concept that frequently differentially expressed mRNA transcripts identified using ADE can be used for the detection of prostate cancer in body fluids such as urine and blood.

Preferred embodiments of the present invention have been disclosed. A person of ordinary skill in the art would realize, however, that certain modifications would come within the teachings of this invention, and the following claims should be studied to determine the true scope and content of the invention. In addition, the methods and compositions of the present invention can be incorporated in the form of a variety of embodiments, only a few of which are described herein. It will be apparent to the artisan that other embodiments exist that do not depart from the spirit of the invention. Thus, the described embodiments are illustrative and should not be construed as restrictive. While the present invention has been particularly shown and described with reference to the preferred and alternative embodiments described herein, it should be understood by those skilled in the art that various alternatives to the embodiments of the invention described herein may be employed in practicing the invention without departing from the spirit and scope of the invention as defined in certain nonlimiting embodiments herein. It is intended that the claims filed herewith define the scope of the invention and that the methods and composition within the scope of these claims and their equivalents be covered thereby. This description of the invention should be understood to include all novel and non-obvious combinations of elements described herein, and claims may be presented in this or a later application to any novel and non-obvious combination of these elements. The described embodiments are illustrative only and do not limit the invention to only those expressly described and do not constitute a disclaimer of other embodiments. No single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

SEQ ID NO: Listing

SEQ ID NO: 1:
KB208E9 mRNA -
CTCTACCTGCATTCCCAAGTAACGGAAAGGAGTAGAGGTTTGAATCTTAT
CAGATGTTATTGTCAGICCCGCGTTGCCAACCTCTGTCTGCAGAAATGTG
TAACGGTCAATTAATTTGTCTCTCGTTTCTGCAGCACACAAAATATCAAC
ATAGTGAACGATGTAACAGTCTGAAAACTTGTCTCTAACTGGTTGCAGAG
CTTGAGCTGACAAATAGTTGAACTATTAAGCATTCCCTGAGGCAATACTT
TCCACTGAAACCTGGT
SEQ ID NO: 2:
KB208E9 cDNA -
ACCAGGTTTCAGTGGAAAGTATTGCCTCAGGGAATGCTTAATAGTTCAAC
TATTTGTCAGCTCAAGCTCTGCAACCAGTTAGAGACAAGTTTTCAGACTG
TTACATCGTTCACTATGTTGATATTTTGTGTGCTGCAGAAACGAGAGACA
AATTAATTGACCGTTACACATTTCTGCAGACAGAGGTTGCCAACGCGGGA
CTGACAATAACATCTGATAAGATTCAAACCTCTACTCCTTTCCGTTACTT
GGGAATGCAGGTAGAGGAAAGGAAAATTAAACCAC
SEQ ID NO: 3:
Forward primer -
5′-TGCCTCAGGGAATGCTTAAT
SEQ ID NO: 4:
Reverse Primer -
5′-CCTCTACCTGCATTCCCAAG
SEQ ID NO: 5:
rp11-442e11 mRNA -
TTACCAGGITGAAATGGGAAACGAGGGAGAAAGGACTTGAAGATGACTCC
AGTGTTTCTAGTAACACAGGTGGTGATGTCACTAATGAGGGTAAAAGCAC
TGAAAGCGCAGGTATGACTTTGGAAAATGGTGGGTTTGAGGTGTTTCTTC
CCAAGCAAGTATTGGGGATTCATGCCAAGAACTTAAGAGTGGTACCAGGG
CCAGACATATAAATTTGGGGTATTTATATCAAATGCTGGTAGAAGTAGCG
AGATTAAAAGAGTTAGCCCTGAGAAAACATAGAGCAAGGAGAGGCAGTTA
AAATCAGCAGAGCCTGCTGAAAAACACCTTCTGTAGAAGGTAG
SEQ ID NO: 6:
rp11-442e11 cDNA -
CTACCTTCTACAGAAGGTGTTTTTCAGCAGGCTCTGCTGATTTTAACTGC
CTCTCCTTGCTCTATGTTTTCTCAGGGCTAACTCTTTTAATCTCGCTACT
TCTACCAGCATTTGATATAAATACCCCAAATTTATATGTCTGGCCCTGGT
ACCACTCTTAAGTTCTTGGCATGAATCCCCAATACTTGCTTGGGAAGAAA
CACCTCAAACCCACCATTTTCCAAAGTCATACCTGCGCTTTCAGTGCTTT
TACCCTCATTAGTGACATCACCACCTGTGTTACTAGAAACACTGGAGTCA
TCTTCAAGTCCTTTCTCCCTCGTTTCCCATTTCAACCTGGTAA
SEQ ID NO: 7:
Forward primer-
5′ -GGTGTTTTTCAGCAGGCTCT
SEQ ID NO: 8:
Reverse primer-
5′ -AAAATGGTGGGTTTGAGGTG
SEQ ID NO: 9:
Forward primer-
5′-GATTTTCACCAATGACCGCCG
SEQ ID NO: 10:
Reverse primer-
5′-CCCCAGCATTGATGTCG
SEQ ID NO: 11:
Forward primer-
5′-CAGGGGAAACAGACGATGACAACT
SEQ ID NO: 12:
Reverse primer-
5′-TGCGGTAACCCAAGCCACACT
SEQ ID NO: 13:
Forward primer-
5′-GAGCCAAAAGTTCTTCTACACTGC
SEQ ID NO: 14:
Reverse primer-
5′-AGATTCCAGATGGTTCTGCCTA
SEQ ID NO: 15:
Forward primer-
5′-GAGATCCCTCCAAAATCAAGTG
SEQ ID NO: 16:
Reverse primer-
5′-CCTTCCACGATACCAAAGTTGT

### REFERENCES

1. Mazzucchelli, R., Colanzi, P., Pomante, R., Muzzonigro. G. & Montironi, R. (2000) Adv Clin Path 4, 111-20.
2. Schamhart, D. H., Maiazza, R. & Kurth, K. H. (2005) Int J Oncol 26, 565-77.
3. Thompson, I. M., Pauler, D. K., Goodman, P. J., Tangen, C. M., Lucia, M. S., Paynes, H. L., Minasian, L. M., Ford, L. G., Lippman, S. M., Crawford, E. D., Crowley, J. J. & Coltman, C. A., Jr. (2004) N Engl J Med 350, 2239-46.
4. Martin, K. J., Graner, E., Li, Y., Price, L. M., Kritzman, B. M., Fournier, M. V., Rhei. E. & Pardee, A. B. (2001) Proc Nati Acad Sci USA 98, 2646-51.
5. Liang, P. & Pardee, A. B. (1998) Mol Biotechnol 10, 261-7.
6. Kim, M. Y., Park, E., Park, J. H., Park, D. H., Moon, W. S., Cho, B. H., Shin, H. S. & Kim, D. G. (2001) Oncogene 20, 4568-75.
7. Chakrabarti, R., Robles, L. D., Gibson, J. & Muroski, M. (2002) Cancer Genet Cytogenet 139, 115-25.
8. Liang. P. & Pardee, A. B. (2003) Nat Rev Cancer 3, 869-76.
9. Patel, K., Whelan, P. J., Prescott, S., Brownhill, S. C., Johnston, C. F., Selby, P. J. & Burchill, S. A. (2004) Clin Cancer Res 10, 7511-9.
10. Zhang, L. & Barritt. G. J. (2004) Cancer Res 64, 8365-73.
11. Tsavaler, L., Shapero, M. H., Morkowski, S. & Laus, R. (2001) Cancer Res 61, 3760-9.
12. Sakko, A. J., Ricciardelli, C., Mayne, K., Suwiwat, S., LeBaron, R. G. Marshall, V. R., Tilley, W. D. & Horsfall, D. J. (2003) Cancer Res 63, 4786-91.
13. Cross, N. A., Chandrasekharan, S., Jokoriya, N., Fowles, A., Hamdy, F. C., Buttle, D. J. & Eaton, C. L. (2005) Prostate 63, 269-75.
14. Boller, K., Konig, H., Sauter, M., Ivlueller-Lantzsch, N., Lower, R., Lower. J. & Kurth, R. (1993) Virology 196, 349-53.
15. Ejthadi, H. D., Martin, J. H., Junying, J., Roden, D. A., Lahiri, M Warren, P., MuiTay, P. G. & Nelson. P. N. (2005) Arch Viral 150, 177-84.
16. Goessl, C., Krause, H., Muller, M., Heicappell, R., Schrader, M., Sachsinger, J. & Miller, K. (2000) Cancer Res 60, 5941-5.
17. Paul, B., Dhir, R., Landsittel, D., Hitchens, M. R. & Getzenberg. R. H. (2005) Cancer Res 65, 4097-100.
18. Tombal, B., Van Cangh, P. J., Loric, S. & Gala, J. L. (2003) Prostate 56, 163-70.
19. Zimmerman, R. A. & Culkin, D. J. (2003) Clin Prostate Cancer 2, 160-6.
20. Wang, Z. P., Eisenberger, M. A., Carducci, M. A., Partin, A. W., Scher, H. I. & Ts'o, P. O. (2000) Cancer 88, 2787-95.
21. Ts'o, P. O., Pannek, J., Wang, Z. P., Lesko, S. A., Bova, G. S. & Partin. A. W. (1997) Urology 49, 881-5.
22. Fehm, T., Sagalowsky, A., Clifford, E., Beitsch, P., Saboorian, H., Euhus, D., Meng, S., Morrison, L., Tucker, T., Lane, N., Ghadimi, B. M., Heselmeyer-Haddad, K., Ried, T., Rao, C. & Uhr, J. (2002) Clin Cancer Res 8, 2073-84.
23. Liana, L. A. & Kohn, E. C. (2003) Nat Genet 33, 10-1
24. Ramaswamy, S., Ross, K. N., Lander, E. S. & Golub, T. R. (2003) Nat Genet 33, 49-54.
25. Chatterjee. S. K. & Zetter, B. R. (2005) Future Oncol 1, 37-50.
26. Tricoli, J. V., Schoenfeldt, M. & Conley, B. A. (2004) Clin Cancer Res 10, 3943-53.
27. Hutchinson, L. M., Chang, E. L., Becker, C. M., Ushiyama, N., Behonick, D., Shih, M. C., DeWolf, W. C., Gaston, S. M. & Zetter, B. R. (2005) Clin Biochem 38, 558-71.
28. Jemal, A., Murray, T., Ward, E., Samuels, A., Tiwari, R. C., Ghafoor, A., Feuer, E. & Thun, M. J. (2005) CA Cancer J Clin 55, 10-30.
29. Scher, H. I. & Heller. G. (2000) Urology 55, 323-7.
30. Magee, J. A., Araki, T., Patil, S., Ehrig, T., True, L., Humphrey, P. A., Catalona, W. J., Watson, M. A. & Milbrandt, J. (2001) Cancer Res 61, 5692-6.
31. Jeronimo, C., Usadel, H., Henrique, R., Oliveira, J., Lopes, C., Nelson, W. G. & Sidransky. D. (2001) J Natl Cancer Inst 93, 1747-52.
32. Dhanasekaran, S. M., Barrette, T. R., Ghosh, D., Shah, R., Varambally, S., Kurachi, K., Pienta, K. J., Rubin, M. A. & Chinnaiyan, A. M. (2001) Nature 412, 822-6.
33. Chaib, H., Cockrell, E. K., Rubin, M. A. & Macoska, J. A. (2001) Neoplasia 3, 43-52.
34. Bubendorf, L., Kolmer, M., Kononen, J., Koivisto, P., Mousses, S., Chen, Y., Mahlamaki, E., Schraml, P., Moch, H., Filli, N., Elkahloun, A. G., Pretlow, T. G., Gasser, T. C., Mihatsch, M. J., Sauter. G. & Kallionienzi, O. P. (1999) J Natl Cancer Inst 91, 1758-64.
35. Singh, D., Febbo, P. G., Ross, K., Jackson, D. G., Manola, J., Ladd, C., Tamayo, P., Renshaw, A. A., D'Amico, A. V., Richie, J. P., Lander, E. S., Loda, M., Kantoff, P. W., Golub, T. R. & Sellers, W. R. (2002) Cancer Cell 1, 203-9.
36. Lapointe, J., Li, C., Higgins, J. P., van de Rijn, M., Bair, E., Montgomery, K., Ferrari, M., Egevad, L., Rayford, W., Bergerheim, U., Ekman, P., DeMarzo, A. M., Tibshirani, R., Botstein, D., Brown, P. O., Brooks, J. D. & Pollack, J. R. (2004) Proc Natl Acad Sci USA 101, 811-6.
37. True, L., Coleman, I., Hawley, S., Huang, C. Y., Gifford, D., Coleman, R., Beer, T. M., Gelmann, E., Datta, M., Mostaghel, E., Knudsen, B., Lange, P., Vessella, R., Lin, D., Hood. L. & Nelson, P. S. (2006) Proc Natl Acad Sci USA 103, 10991-6.
38. Weinberg, R. A. (2007) in The biology of cancer (Garland Science, New York), pp. 587-654.
39. Menke, T. B. & Warnecke, J. M. (2004) Ann N I Acad Sci 1022, 185-9.
40. Altschul, S. F., Madden, T. L., Schaffer, A. A. Zhang, J., Zhang, Z., Miller. W. & Lipman, D. J. (1997) Nucleic Acids Res 25, 3389-402.

What is claimed is:

1. A method for assessing the presence of prostate cancer in a human, comprising, the steps of:
providing, a sample of prostate tissue, blood, or urine from a human; and
determining the level of SEQ ID NO: 1 in the sample, wherein an elevated level of SEQ ID NO: 1 in the sample is indicative of the presence of prostate cancer in the human.

2. The method of claim 1, wherein the determining step comprises determining the level of SEQ ID NO: 1 by a reverse transcriptase polymerase chain reaction assay.

3. The method of claim 2, further comprising the step of determining the level of expression of a constitutively expressed housekeeping gene.

4. The method of claim 1, wherein the sample is a prostate tissue sample, and further comprising the step of comparing the determined level of SEQ ID NO: 1 in the sample to a value for the level of SEQ ID NO: 1 derived from other human prostate tissue samples.

5. The method of claim 1, wherein the sample is a blood or urine sample, and further comprising the step of comparing the determined level of SEQ ID NO: 1 in the sample to a value for the level of SEQ ID NO: 1 derived from human blood or urine samples.

6. A method for assessing, the presence of prostate cancer in a human, comprising the steps of:
providing a sample of prostate tissue, blood, or urine from a human; and
determining the level of SEQ ID NO: 5 in the sample, wherein a reduced level of SEQ ID NO: 5 in the sample is indicative of the presence of prostate cancer in the sample.

7. The method of claim 6, wherein the determining step comprises determining the level of SEQ ID NO: 5 by a reverse transcriptase polymerase chain reaction assay.

8. The method of claim 7, further comprising the step of determining the level of expression of a constitutively expressed housekeeping gene.

9. The method of claim 6, wherein the sample is a prostate tissue sample, and further comprising the step of comparing the determined level of SEQ ID NO: 5 in the sample to a value for the level of SEQ ID NO: 5 derived from other human prostate tissue samples.

10. The method of claim 6, wherein the sample is a blood or urine sample, and further comprising the step of comparing the determined level of SEQ ID NO: 5 in the sample to a value for the level of SEQ ID NO: 5 derived from other human blood or urine samples.

11. A method for assessing the presence of prostate cancer in a human, comprising the steps of:
providing a sample of prostate tissue, blood, or urine from a human;
determining the level of SEQ ID NO: 1 in the sample;
determining the level of SEQ ID NO: 5 in the sample; and
determining the ratio of the level of SEQ ID NO: 1 in the sample to the level of SEQ ID NO: 5 in the sample, wherein an increase in the ratio is indicative of the presence of prostate cancer in the human.

12. The method of claim 11, wherein the determining steps comprise determining the level of SEQ ID NO: 1 or SEQ ID NO: 5 by a reverse transcriptase polymerase chain reaction assay.

13. The method of claim 12, further comprising the step of determining the level of expression of a constitutively expressed housekeeping gene.

14. The method of claim 12, wherein the sample is a prostate tissue sample, and further comprising the step of comparing the determined levels of SEQ ID NO: 1 and SEQ ID NO: 5 in the sample to values for levels of SEQ ID NO: 1 and SEQ ID NO: 5 derived from other human prostate tissue samples.

15. The method of claim 12, wherein the sample is a blood or urine sample, and further comprising the step of comparing the determined levels of SEQ ID NO: 1 and SEQ ID NO: 5 in the sample to values for level of SEQ ID NO: 1 and SEQ ID NO: 5 derived from other human blood or urine samples.
